(19) United States
(12) Patent Application Publication
(10) Pub. No.: US 2023/0305005 A1
(43) Pub. Date: Sep. 28, 2023

(54) CORONAVIRUS POINT-OF-CARE AGGLUTINATION ASSAY

(71) Applicant: Veravas, Inc., Austin, TX (US)

(72) Inventors: Joshua Caine Soldo, Prior Lake, MN (US); Scott Douglas Bergmann, River Falls, WI (US)

(21) Appl. No.: 18/041,025

(22) PCT Filed: Aug. 16, 2021
(86) PCT No.: PCT/US21/46177

§ 371 Date: Feb. 8, 2023

Related U.S. Application Data
(60) Provisional application No. 63/065,993, filed on Aug. 14, 2020.

(51) Int. Cl. G01N 33/569; G01N 33/543; C07K 16/10

(52) U.S. Cl. CPC G01N 33/56983; G01N 33/54326; C07K 16/10; G01N 2469/10; G01N 2333/165

(57) ABSTRACT

The present specification provides reagents and methods for a rapid detection of SARS-CoV-2. The assay can be implemented in a point-of-care or laboratory setting.

### CROSS REFERENCE TO RELATED APPLICATIONS

The present application claims the benefit of U.S. Provisional patent application 63/065,993 filed Aug. 14, 2020, which is incorporated by reference herein in its entirety.

### BACKGROUND

There are many assay technologies that have been utilized for detecting viral infection in individuals. While a variety of assays for SARS-CoV-2, the causative agent of COVID-19, have been and are being developed, many of these require a laboratory, complex equipment, personnel with specialized training, and substantial time to carry out the assay. These requirements limit the usefulness of such assays for rapid identification of infected individuals who could potentially transmit COVID-19 at public gatherings.

### SUMMARY

There is a need for a point-of-care (POC) assay for the detection and/or quantitation of SARS-CoV-2 virus, the causative agent of COVID-19. Disclosed herein are reagents and methods for a rapid, POC assay, that can be conducted wherever a potentially infected individual may be encountered, where the assay can be completed in a few minutes (e.g., 5 minutes), and produces a visually detectable result. The assay requires only a saliva sample from the subject being tested.

In further embodiments the assay procedure can be adapted for use in a clinical laboratory in which multiple samples are processed in parallel, for example in a 96-well plate, so as to enable high-throughput. In one aspect of these embodiments, the amount of the virus in the sample can be quantitated.

The disclosed assays make use of beads coated with anti-SARS-CoV-2 surface antigen antibody which undergo agglutination is the presence of SARS-CoV-2. In some embodiments, the antibody recognizes the S1 or S2 spike protein of SARS-CoV-2. In some embodiments, the antibody recognizes the receptor binding domain (RBD) of the S1 spike protein. In some embodiments, the antibody recognizes the N-terminal domain (NTD) of the S1 spike protein. In some embodiments, the antibody is a neutralizing antibody. In other embodiments, the surface antigen recognized by the antibody is the hemagglutinin protein, matrix (M) protein of envelope (E) protein. In some embodiments, the neutralizing antibody has an IC50 of 3-4 nM by competitive ELISA.

In some embodiments, the beads are latex beads. In some embodiments, the beads are magnetic beads. In some embodiments, the beads have a dark or intense color.

In some embodiments, the beads are fluorescent. In some embodiments the beads are tagged with luciferase or other luminescence-generating agent. In still other embodiments, the bead is tagged with any other visually- or spectrophotometrically-detectable signal generating agent.

In some embodiments, the beads have a diameter of from 1.3 to 1.9 μm or from about 0.55 μm to about 2.7 μm. In some embodiments, the beads have a diameter of about 550 nm. In some embodiments, the beads have a diameter of about 1.6 μm. In some embodiments, the beads have a diameter of about 2.7 μm. These diameter sizes refer to the diameter of the beads prior to adding streptavidin, antibody, or other coatings or modifications.

In some embodiments, the beads are streptavidinated. In some embodiments, the beads are carboxy beads and the streptavidin is covalently attached to the beads using 1-ethyl-3-(3-dimethylaminopropyl)carbodiimide (EDC) chemistry. In some embodiments, the antibody is biotinylated and coated onto streptavidinated beads through biotin-streptavidin binding. In some embodiments, the antibody coating comprises from 25 to 35 μg of antibody/mg of beads. In some embodiments, the antibody coating comprises 30 μg of antibody/mg of beads.

In some embodiments, the anti-SARS-CoV-2 antibody is conjugated to the bead with another affinity reagent than streptavidin. In various embodiments, the conjugating affinity reagent is protein A, protein G, and anti-Fc antibody, an anti-species Ig antibody (for example, goat anti-rabbit Ig or rabbit anti-mouse Ig), or an anti-label antibody (for example, an antibody recognizing biotin, fluorescein, dextran, etc.) depending on the nature of anti-SARS-CoV-2 antibody and what it may have been modified with.

In some embodiments, antibody is directly attached to a carboxy bead using EDC chemistry. In some embodiments, antibody is directly attached to tosyl activated beads or to epoxy activated beads.

The basic assay procedure comprises combining anti-SARS-CoV-2 surface antigen antibody-coated beads with a saliva sample from a subject to be tested for SARS-CoV-2 infection; mixing the beads and the saliva sample to form an admixture; incubating the admixture; and detecting whether agglutination has occurred. In some embodiments, the incubation is for about 5 minutes. In some embodiments, the incubation is at room temperature. In other embodiments the incubation is at 37° C.

In a variation on the basic assay procedure, anti-SARS-CoV-2 surface antigen antibody-coated beads are combined with a saliva sample from a subject to be tested for SARS-CoV-2 infection in a multiwell plate; the beads are incubated at room temperature for 1-10 minutes (for example, 5 minutes); and the plate is placed in a plate shaker for an interval of time. In some embodiments, the interval is 5 minutes. In some embodiments, the shaker is at room temperature. In other embodiments the plate shaker is at 37° C. In some embodiments, a linear shaking motion is used. In some embodiments, the shaking is fast, for example, about 1000 cycles/minute.

In some embodiments, the saliva sample is comprised in an oral rinse fluid. The oral rinse fluid can be obtained by swishing and gargling with a saline solution, for example, a 0.9% saline solution, and expectorating into a collection vessel. In some embodiments, 5 mL of saline solution is used for the rinse. In some embodiments, swishing and gargling proceeds for 30 seconds.

Alternatively, in some embodiments, a nasal swab or nasopharyngeal swab specimen is collected in a saline solution or other transport media by agitation of the swab in the fluid to disperse any virus present in the specimen. In another variation the swab is agitated in oral rinse solution containing a saliva sample, so that virus present in either can be detected. The transport media can be UTM®, Universal Transport Medium™ (Copan Diagnostics, Inc. Murrieta, CA), a room temperature stable viral transport medium for collection, transport, maintenance and long term freeze storage of viruses and other infectious specimens, consisting of Hank's balanced salt solution, bovine serum albumin, L-cysteine, gelatin, sucrose, L-glutamic add, HEPES buffer, phenol red, sucrose, vancomycin, amphotericin B, and colistin. The medium is isotonic and non-toxic to mammalian host cells.

In some embodiments, each individual's saliva sample is processed separately from collection through detection. In some embodiments, the admixtures, or aliquots thereof, from multiple individuals are transferred to separate wells of a multiwell plate after the mixing or incubating step, and detection takes place in a microplate reader or microarray digital reader. In some embodiments, a viscosifying agent is added to the admixtures after the incubating step, but before the transferring step. In some embodiments, the viscosifying agent is FICOLL.

In some embodiments, known quantities of virus, for example, a dilution series, are assayed to generate a calibration curve from which the amount of virus in an individual's saliva sample can be quantitated.

In some embodiments, the assay is qualitative, discriminating between the presence and absence of virions, but not providing quantitation of the number of virions present. In some embodiments, the qualitative assay can detect at least as few as 100 virions per milliliter. In some embodiments, the qualitative assay can detect at least as few as 10 virions per milliliter.

### BRIEF DESCRIPTION OF DRAWINGS

FIG. 1 displays assayed saliva samples that were polymerase chain reaction (PCR)-positive (left) or PCR-negative (right) for SARS-CoV-2. Bead-based agglutination is clearly visible in the vial on the left (the virus-containing sample), whereas no bead agglutination is observed in the vial on the right (the sample without virus).

FIGS. 2A-D depict the change in interference (in nanometers) as RBD polypeptide binds to and dissociates from antibody immobilized on a bio-layer interferometry sensor. The vertical dotted line at 0 seconds indicates the time the biosensor was immersed in a solution of RBD polypeptide and the vertical dotted line at 240 seconds indicates the time at which the biosensor was removed from the RBD polypeptide solution and immersed in buffer. Each of the plots show three pairs of tracings. Each pair represent the actual data and a fitted curve from which kinetic constants (KD, Ka, and Kd) where derived. For several of the pairs the tracings for the data and the fit were indistinguishable. In each plot the uppermost tracings are for 100 nM RBD polypeptide, the intermediate tracings were for 10 nM RBD polypeptide, and the bottom tracings for 0 nm RBD polypeptide. 2A-Ty1; 2B-MM57; 2C-R001; and 2D-MM43

FIG. 3 shows images of agglutination reactions for Ty1-coated 550 nm beads incubated with SARS-CoV-2 negative (top) and positive (bottom) samples from Example 5.

FIG. 4 shows images of agglutination reactions for R001-coated 2.7 μm beads incubated with SARS-CoV-2 negative (top) and positive (bottom) samples from Example 5.

FIG. 5 shows images of the agglutination reactions described in Example 6. There are five images, from left to right, beads only, FIG. 7 negative sample +100 μL of beads, 100 μL of positive sample +50 μL of beads, 100 μL of positive sample +100 μL of beads, and 100 μL of positive sample +200 μL of beads.

FIGS. 6A-B show images of the agglutination reactions described in Example 7. An array of four wells is seen. The upper wells contain the negative sample and the lower well the positive sample. The left pair of wells held the agglutination reaction using the 2:1 ratio of bead to sample, and the right pair of wells held the agglutination reaction using the 1:1 ratio of bead to sample. 6A shows the raw image and 6B shows the same image after processing.

FIG. 7 show images from the bead dilution agglutination assay described in Example 8. The dilution proceeds from left to right in the ratio of 100:75:50:25:10:0. The upper row received the negative sample and the lower row received the positive sample.

FIG. 8 is a diagram of a point-of-care device for carrying out a SARS-CoV-2 agglutination assay.

FIGS. 9A-B. A test card for a smartphone-based point-of-care SARS-CoV-2 agglutination assay is depicted in 9A. The test card shows appropriate positioning of a test chamber and provides an appropriate background for image capture. 9B depicts mock-up result screens from an image reading smartphone app.

FIGS. 10A-C portray SARS-CoV-2 assay results. 10A reports the object sum area (OSA) from saliva samples from infected and uninfected persons, titrated virions, and saline, as well as signal to noise ratios (S/N) for each positive sample over the average of the three negative samples at the 15 minute endpoint and for the slope over 0 to 3 minutes. 10B is a plot of virion count versus the clump count reading over 0 to 3 minutes. 10C is a plot of virion count versus the slope of the clump count reading at 15 minutes. 10B-C plot only the three negative samples (A) and the serial dilutions of virions (•).

FIG. 11 presents an overview of one embodiments of a complete agglutination test process.

FIG. 12 shows a saliva device for collecting a saline oral rinse to be tested in certain embodiments.

FIGS. 13A-B present results from an agglutination assay interpreted by the slope of OSA change. 13A plots OSA over time for each of the samples tested, labeled by sample number. 13B presents images of each sample. See Table 4 for sample numbers and descriptions.

### DESCRIPTION

One simple technology for detecting, or quantitating, a polyvalent analyte such as a virus particle, is agglutination. Agglutination entails the clumping together of particles, typically depending upon antibody-antigen binding. Classic examples of agglutination-based assays include ABO blood typing and the Monospot assay for Epstein-Barr virus infection. Agglutination can occur when the antibody component of the reaction can form cross-links between two (or more) antigen-bearing particles and each antigen-bearing particle is cross-linked to multiple other particles. If there is too much antibody present relative to antigen, the antibody will saturate all of the binding sites on the antigen bearing particle so that, effectively, no cross-linking takes place and agglutination does not occur. Steric factors can also interfere with agglutination if, for example, the geometry of binding is such that an antibody bound to one antigen-bearing particle cannot reach across to a second antigen-bearing particle, or if the kinetics of binding are such that both binding sites of the antibody (assuming, for example, a bivalent antibody) engage the same antigen-bearing particle. Steric factors such as these can often be overcome by attaching antibody to beads, effectively increasing their valency, reach, and positional diversity.

Attaching the antibody to beads also addresses a further issue. Virus particles and antibody molecules are so small that even if agglutination does occur, the agglutinate (the clump) may not be visually observable. By attaching the antibody to beads which are macroscopically visible, at least when agglutinated, a successful agglutination reaction can be detected by eye (or camera).

This agglutination method detects virions and not RNA or antigen dissociated from the virion. Agglutination will occur best with intact virion samples such as fresh saliva samples, oral saline rinse samples, or swab/oral saline rinse samples, as well as gamma irradiated saliva-based samples. Heat inactivation may denature or break down the virion structure for some virions which will decrease agglutination efficiency for damaged virions or virion fragments. As agglutination depends on the virions being substantially intact, it will be less prone to false positive tests arising from residual antigen and RNA after an infection has resolved. This contrasts with ELISA- and PCR-based tests which detect antigen and RNA, respectively, whether or not they are associated with an intact virion.

In particular embodiments, an anti-SARS-CoV-2 surface antigen antibody is biotinylated and is reacted with streptavidinated, magnetic beads. Several of these features are convenient, but not essential. The biotin-avidin reaction is widely used for attaching antibodies to beads and other substrates, the reaction is well-understood, and the necessary reagents are readily available. Nonetheless, other chemistries for attaching an antibody to a bead are known and could be utilized. Similarly, magnetic beads can be easy to process, but the general assay protocols herein disclosed do not make use of magnetism. However, magnetic beads typically contain iron and have a dark brown color which facilitates visual detection. Non-magnetic beads can also be used, but should be of a dark and/or intense color that will facilitate visual detection. In addition to brown, black and darker shades of blue, green, red, and purple are appropriate, whereas white, yellow and tan, for example, would be less preferred. Latex beads have often been used in agglutination assays and are available in multiple colors.

In particular embodiments, the anti-SARS-CoV-2 surface antigen antibody is a neutralizing antibody that binds to the receptor binding domain of the SARS-CoV-2 spike protein. The neutralizing activity of the antibody is not essential to the assay. Any antibody that binds to a multivalent site on the virus particle is potentially useful. However, the receptor binding domain is an accessible and well-conserved site making it well-suited for the present purpose. Monoclonal antibodies recognizing the SARS-CoV-2 S1 and S2 spike proteins and the receptor binding domain, including neutralizing monoclonal antibodies, from mouse and rabbit are commercially available from multiple sources. Neutralizing single domain antibody fragments from alpaca have also been generated (Hanke et al., bioRxiv 2020.06.02.130161, which is incorporated herein by reference in its entirety). While these antibody fragments (nanobodies) are monovalent, they are nonetheless suitable for use in an agglutination assay when attached to beads, as the beads will be multivalent.

The alpaca nanobody (Ty1) offers several advantages. The nanobody is enzymatically biotinylated on the C-terminus using Sortase A, which assures a consistent orientation of the nanobody to the bead with the antigen binding site facing away from the bead, which is optimal for virus binding. This contrasts with randomly biotinylated antibodies where the antibody is attached to the bead in a variety of orientations, some of which will be sterically hindered. Ty1 is also able to recognize the RBD in both its “up” and “down” conformation (also referred to as “open” and “closed” conformations, respectively). The small size of the nanobody (only 12.5 kD) allows a nanobody to bind to the RBD in each of the three S1 protomers in one spike protein. The small size of the nanobody also means that, per mg of antibody coating a bead, more virus binding sites are present that with full size antibodies.

Antibody affinity is also an important parameter to consider in choosing an antibody to use as an agglutination reagent, with higher affinity being associated with more robust agglutination. In some embodiments, the antibody has a KD that is greater than 2.5, 5.0, 7.5, 9.0, or 9.5, as measured in phosphate buffered saline (PBS) by biolayer interferometry. In various embodiments, the KD is in a range of from 2.5, 5.0, 7.5, 9.0, or 9.5 to 10. In some embodiments, the KD is about 2.8. In some embodiments, the KD is about 9.9.

In particular embodiments, magnetic beads with free carboxylate groups (carboxy beads) are covalently linked to streptavidin using 1-ethyl-3-(3-dimethylaminopropyl)carbodiimide (EDC) chemistry, blocked and stripped to prevent non-specific binding. A biotinylated antibody recognizing SARS-CoV-2 is then attached utilizing biotin-streptavidin binding. Procedures and variations for this steps are known in the art (see for example PCT/US2020/039503, which is incorporated herein by reference for all that it teaches about making and using beads coated with streptavidin and attaching biotinylated molecules). It is desirable that the streptavidin coated beads, and subsequent biotin-antibody coated streptavidin beads, are monodisperse, both to maximize binding surface area, and the assay is based on bead aggregation/agglutination. Vigorous bead mixing, shear mixing, and sonication can be used to ensure the beads are mixed, homogeneous, and monodisperse. Monodisperse beads (non-aggregated beads) also give the optimal control result with PCR negative saliva samples, as the beads should not aggregate or agglutinate in the absence of their viral target.

In some embodiments, antibody is directly attached to a carboxy bead using EDC chemistry. In some embodiments, antibody is directly attached to tosyl activated beads or to epoxy activated beads. In still other embodiments, the antibody is coated into the bead non-covalently using coordinated chemistry bonding.

Embodiments utilizing streptavidin are described throughout this disclosure. However, further embodiments comprising alternatives, such as avidin, deglycosylated avidin (neutravidin), CaptAvidin, monomeric avidin, are also contemplated. Natural and recombinant versions of streptavidin, and its alternatives, are also contemplated. These reagents may be referred to as means for binding biotin.

Instead of streptavidin or its analogs, other affinity reagents may be attached to the bead and used for conjugation of the anti-SARS-CoV-2 antibody. In some embodiments, the conjugating affinity reagent, is an anti-biotin antibody and can conjugate a biotinylated anti-SARS-CoV-2 antibody. In some embodiments, the conjugating affinity reagent recognizes a label such as fluorescein, dextran, His-tag, etc., and can conjugate a fluoresceinated, dextran-modified, His-tagged, or otherwise labeled anti-SARS-CoV-2 antibody, respectively. In some embodiments, the conjugating affinity reagent recognizes the Fc region of an antibody, such as protein A, protein G, or an anti-Fc antibody, and can conjugate an anti-SARS-CoV-2 antibody that comprises an Fc region. In some embodiments, the conjugating affinity reagent is an antibody that recognizes species-specific epitopes in an immunoglobulin (Ig), for example, a goat anti-rabbit Ig antibody or a rabbit anti-mouse Ig antibody, and can conjugate an anti-SARS-CoV-2 antibody that is a rabbit or mouse antibody, respectively.

Beads of various sizes may be used. In some embodiments, the beads have a diameter of from about 500 nm to about 2.7 μm, or from about 1.3 to about 1.9 μm. In some embodiments, the beads have a diameter of 1.6 μm. In some embodiments, the beads have a diameter of 2.7 μm. Bead size can effect settling time, with larger beads settling faster. In flat-bottom 96-well plates containing 100-200 μL of liquid (without a viscosifying agent), settling of 2.7 μm beads can be complete in about 3 minutes and settling of 1.6 μm beads can be complete in about 5 minutes. 550 nm beads can take longer to settle. However, as beads agglutinate/aggregate they become very large clumps which can decrease settling time for the agglutinate as compared to the monodisperse beads.

Beads may be coated with various amounts of antibody. In some embodiments, the beads contain from about 10 to about 50 μg of antibody/mg of bead, or from about 25 to about 35 μg of antibody/mg of bead. In some embodiments, the beads contain 30 μg of antibody/mg of bead.

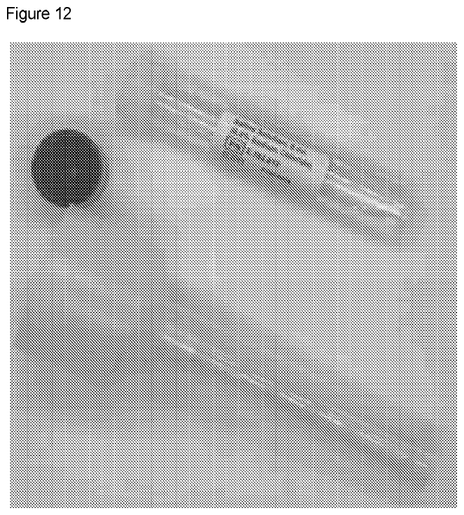

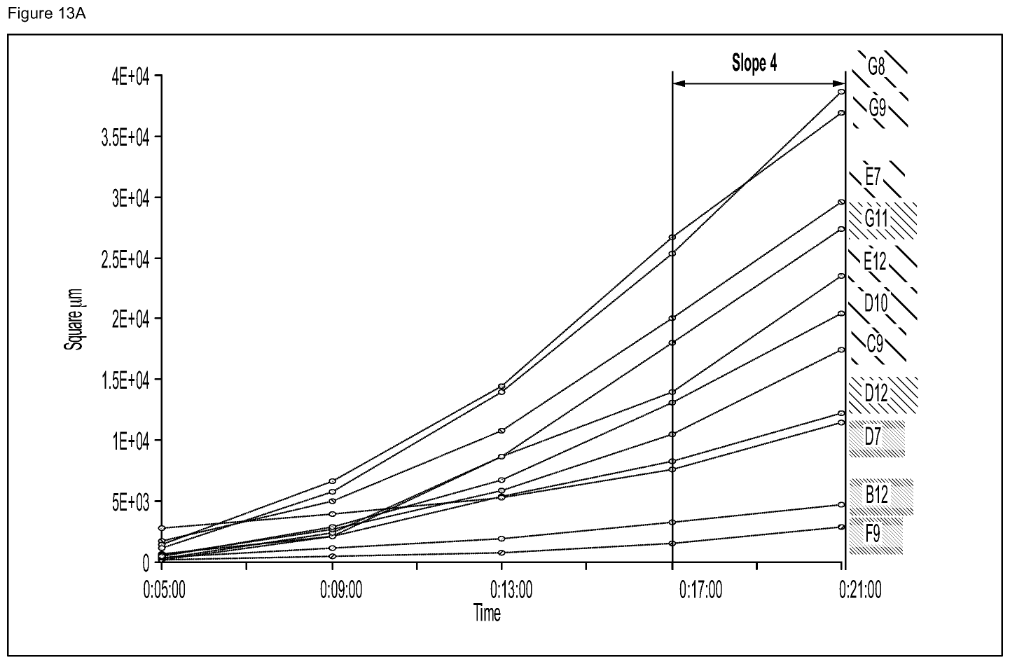

In some embodiments, to obtain a saliva sample to assay, the oral cavity is rinsed with a saline solution and the rinse liquid collected (swish and spit) to obtain an oral rinse fluid. In some embodiments, 5 mL of saline solution is vigorously swished and gargled for 30 seconds and then expectorated into a collection vessel, for example a funneled collection tube. (The universal oral rinse collection kit from OralDNA Labs is suitable for this purpose; FIG. 12).

In some embodiments, the saliva sample, however obtained, is centrifuged to remove cellular material.

To conduct an assay a suspension of beads is added to a saliva sample. In some embodiments, the saliva sample is comprised in an oral rinse fluid. In some embodiments the components are combined at a ratio of 1:2 beads:saliva sample, using a 1 mg/mL suspension of anti-SARS-CoV-2 antibody-coated beads. In aspects of these embodiments the concentration of the bead suspension can be more or less than 1 mg/ml, depending on the desired total reaction volume and dilution factor due to the saliva sample. In some embodiments, 0.5 ml of bead suspension is added to 1 mL of saliva sample (e.g., oral rinse fluid). Other embodiments use other bead:sample ratios and bead concentrations (see Examples 6 and 8). The combined bead suspension and saliva sample are mixed and then incubated. In some embodiments, the incubation is at room temperature. In other embodiments the incubation is at 37° C. In some embodiments, the incubation is for at least 1, 2, 3, 4, or 5 minutes, for 5 to 15 minutes, for 2 minutes, for 3 minutes, or for 5 minutes. In some embodiments, incubation for agglutination and settling take place in a single step.

Generally, it is important to maintain an optimized ratio of bead (agglutination reagent) to saliva sample volume. This can be scaled for larger or smaller sized reactions, for example, reactions carried out in glass vials versus microtiter plates. However, detection sensitivity can be increased by using larger volumes of saliva sample (and proportionally more agglutination reagent) as there will be a greater mass of material from which to form agglutinate.

In an alternative embodiment, when magnetic beads are used, the beads are magnetically separated from the suspending fluid and then resuspended in the saliva sample (oral rinse fluid). The final concentration of beads can be similar to that described above, for example, about 0.33 mg beads/mL. In various embodiments, the bead concentration in the agglutination reaction can be from 0.1 to 1.5 mg/mL, for example, 0.13, 0.33, 0.5, 0.67, 0.75, 1.0, or 1.33 mg/mL, or any range bounded by a pair of these values. The resuspended beads are then incubated, in some embodiments at room temperature or 37° C. In some embodiments, the incubation is for at least 1, 2, 3, 4, or 5 minutes, for 5 to 15 minutes, for 2 minutes, for 3 minutes, or for 5 minutes, by which time agglutination has occurred.

In further embodiments, there is no explicit mixing prior to incubation, beyond that due to the addition of the components. In further embodiments, static incubation is followed by incubation on a shaker, for example a plate shaker. In some embodiments, the shaking incubation is at room temperature, while in others it is at elevated temperature, for example 37° C. In some of these embodiments, the shaking incubation is preceded by a static incubation, for example about 1 minute, or about 5 minutes. In other embodiments there is no explicit static incubation following completing distributing the reagents to the plate.

This agglutination is a visible event and can be read by a variety of methods, ranging from human visual assessment all the way to fully automated high-throughput plate imagers. In many embodiments it is sufficient to visually assess (by eye) the formation of agglutinates. In alternative embodiments, agglutinates can be detected with a camera (for example, a smart phone camera), an optical density reader, a spectrophotometer, a luminometer, a fluorimeter, or a digital flow cell, a particle sizer (e.g., the Anton Paar Litesizer 500). All of these modes of detecting agglutinates can be referred to as a step for detecting agglutination.

For higher throughput applications, an aliquot of the agglutination reaction (i.e., bead and saliva sample admixture) can be transferred to the well of a multiwell plate, for example a 96-well plate, after the mixing or incubation step. Detection of agglutinate formation can be accomplished with a microplate reader, by optical density, for example, or with a microarray digital reader, by image analysis. These modes of detecting agglutinates can also be referred as a step for detecting agglutination, or as a high throughput step for detecting agglutination. In such embodiments, it is also possible to include a dilution series of a known virus sample as a calibration curve so that the amount of virus present in the saliva sample can be quantitated.

Beads and agglutinates will both have a tendency to settle. In some embodiments, this can be undesirable. To inhibit settling, a viscosifying agent can be added, with mixing, to the agglutination reaction after the incubation step and before a detection step, and before a transfer step, if one is used. One suitable viscosifying agent is FICOLL (a neutral, highly branched, high-mass, hydrophilic polysaccharide).

Microarray digital readers are camera based and use image analysis. They have a narrow depth of focus. This contrasts with a microplate reader which operates by passing a beam of light through the entire depth of a sample and measuring optical density, transmission or absorbance (though some readers take measurements at multiple spots in the sample). As a result of the narrow depth of focus, a microarray digital reader can, and often does, look just at the bottom of the well, in which case settling of the agglutinates would be desirable. However, it is also possible to change the plane of focus and scan through the entire depth of a sample to build a 3-dimensional image. This process typically takes little more than one minute per plate. When scanning through multiple planes of focus, the use of a viscosifying agent to inhibit settling of the beads and agglutinates can be desirable. A more even distribution of beads and agglutinates may also be advantageous when using a microplate reader, so a viscosifying agent can be used in such embodiments as well.

Some microplate readers are capable of scanning across a well to produce a 2-dimensional (object sum area) or 3-dimensional profile from which the surface area or volume of the detected peaks and troughs can be calculated, and used to quantitate agglutination. In some embodiments, the assay result is based on the reading (e.g., volume, surface area, or object sum area) at a specific time point after beginning the assay, for example, 2-20 minutes or any integer value in that range, such as at 15 minutes. In some embodiments, assay result is based on the rate of change of the reading (the slope) over a time interval from time zero to 2-20 minutes or any integer value in that range, such as 0 to 3 minutes.

There herein described assays can be used to detect SARS-CoV-2 infection for any purpose. However, they are well suited to circumstances in which speed and/or simplicity are advantageous or required. For example, these assays can be used for home screening, for example before returning to work after a SARS-CoV-2 infection, or for screening to identify persons prior to allowing them to enter any place of public gathering, be that a school, a government office, a house of worship, a shop, a sporting event, an airport or airline flight, etc.

When using magnetic beads, confirmatory testing of positive results by PCR can be carried out on the assayed sample itself. Reagents to extract the viral RNA are added to the well or vial, the beads are magnetically separated, and the fluid is transferred to a PCR reaction. Confirmatory PCR testing can also be used to confirm that the virus detected is in fact SARS-CoV-2, and not a cross-reacting corona virus, or to identify what strain of SARS-CoV-2 is present.

Alternatively, the agglutination reagent can be used to clean a saliva sample prior to a PCR test when the presence or potential presence of interfering substances in the saliva is an issue. The anti-SARS-CoV-2 antibody coated magnetic beads are added to a saliva sample and incubated to bind virus. The beads (agglutinated or not) are magnetically separated and washed. The viral RNA is then extracted as usual, the beads again magnetically separated and the viral RNA-containing fluid collected for PCR analysis.

### EXAMPLES

The following non-limiting examples are provided for illustrative purposes only in order to facilitate a more complete understanding of representative embodiments now contemplated. These examples should not be construed to limit any of the embodiments described in the present specification.

### Example 1

### Bead Preparation

Magnetic beads with diameters of 550 nm and 1.6 μm, bearing free carboxyl groups, were coated with covalently-attached streptavidin as follows:
1. EDC (1-Ethyl-3-(3-dimethylaminopropyl)carbodiimide) chemistry was used to coat the 1.6 um and 550 nm carboxy beads with Streptavidin.
2. 40 mM MES, pH 5.2, was the coupling buffer.
3. 0.5 mg of Streptavidin was used per 1.0 mg of beads during coupling
4. 0.1 mg of EDC was added per 1.0 mg of beads (18.6 mol EDC per mol COOH on 1.6 um beads; 9 mol EDC per mole COOH on 550 nm beads). The EDC was added in 40 mM MES solution, pH 5.2, at 10 mg/mL.
5. The reaction was carried out at a bead concentration of 10 mg/mL.
6. The EDC reaction was allowed to mix at RT for about 12 hours
7. The beads were stripped of passively adsorbed streptavidin to ensure covalent attachment, and blocked with polymeric blockers to decrease non-specific binding by the beads and ensure monodispersity.
8. The beads were washed into TBS with Tween-20 and NaN₃ (10 mM Tris, 150 mM NaCl, 0.05% Tween-20, 0.05% NaN₃) at a 10.0 mg/mL bead concentration.

Three anti-SARS-CoV2 neutralizing antibodies, which recognize the receptor binding domain of the SARS-CoV2 spike protein, were biotinylated as follows:
1. The three antibodies were brought to 1 mg/mL in PBS (40 mM Na₂HPO₄, 10 mM KH₂PO₄, 137 mM NaCl, 3 mM KCl) pH 8.0 in a total volume of 100 μL (100 μg of antibody).
   a. Sino Biological Cat. No. 40591-MM43 lot HB14AP2001, 100 μg, 1.63 mg/mL in PBS (comp. unk.). Mouse monoclonal antibody with typical IC₅₀ of 1.41 μg/mL by a neutralization assay, and 0.857 nM by an ELISA, as reported by the manufacturer.
   b. Sino Biological Cat. No. 40592-MM57 lot HB14AP2002, 100 μg, 1.98 mg/mL in PBS (comp. unk.). Mouse monoclonal antibody with typical IC₅₀ of 0.41 μg/mL by a neutralization assay, and 3.694 nM by an ELISA, as reported by the manufacturer.
   c. Sino Biological Cat. No. 40592-R001 lot HA14MY2101, 100 μg, 5.36 mg/mL in PBS (comp. unk.). Rabbit monoclonal antibody with typical IC₅₀ of 0.11 μg/mL by a neutralization assay, and 0.59 nM by an ELISA, as reported by the manufacturer.
2. QUANTA Biotin-dPEG₄-TFP Ester Cat. No. 10009 lot AF1-A0402-011 was reconstituted in DMSO (LT Baker #9224-01 lot 0000217025) and added to the Antibody solutions.
3. A 10× molar excess of biotin linker to antibody was used.
4. The incubation time was 2 hours at RT on a rotating mixer.
5. The biotinylated antibody was not desalted post-biotinylation.
6. The biotinylated antibody was coated onto Streptavidin coated beads of three sizes:
   a. 550 nm—the MM43 antibody was coated at 50 μg antibody per mg of beads
   b. 1.6 μm—the MM57 antibody was coated at 30 μg antibody per mg beads
   c. 2.7 μm the R001 antibody was coated at 10 μg antibody per mg beads The 550 nm and 1.6 μm beads were prepared as described above. The 2.7 μm bed were purchased in streptavidinated form (Agilent PN 6727-1003; 2220 picomoles per mg bead binding capacity (Biotin-4-Fluorescein binding capacity)), but were stripped and blocked as described above.
7. The beads were coated at a concentration of 10 mg/mL.
8. The incubation time of antibody and beads was 2 hours at room temperature on a rotating mixer.
9. The beads were washed (by magnetic separation) three times into TBS with TWEEN-20 and NaN₃ (10 mM Tris, 150 mM NaCl, 0.05% Tween-20, 0.05% NaN₃) at a 1.0 mg/mL bead concentration.

### Example 2

### Agglutination

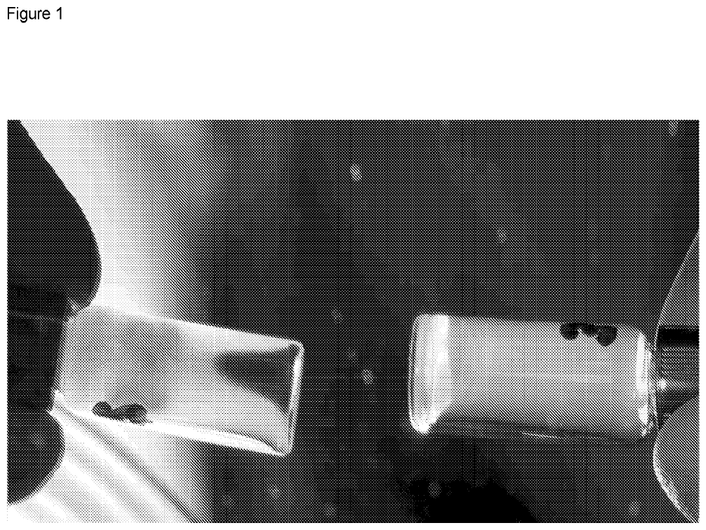

0.5 mL of a 1 mg/mL suspension of 1.6 μm diameter streptavidinated magnetic beads coated with 30 μg/mg MM57 antibody was added to 1 mL each of SARS-CoV-2 PCR-positive and -negative oral rinse. The combined reagents were mixed and incubated at RT for 5 minutes. Agglutinated beads were visually apparent in the virus positive sample, but not the virus-negative sample (FIG. 1).

### Example 3

### Determination of Antibody Dissociate Constants

Bio-layer intereferometry (BLI) was used to determine binding affinity with the RBD portion of the SARS-CoV-2 S1 spike protein for four antibodies: MM57, R001, MM43, and Ty1, an alpaca-derived nanobody, as described above. MM57, R001, and MM43 were biotinylated at random lysines, while Ty1 was biotinylated at a unique C-terminal site, assuring uniform orientation with respect to the substrate to which it is attached.

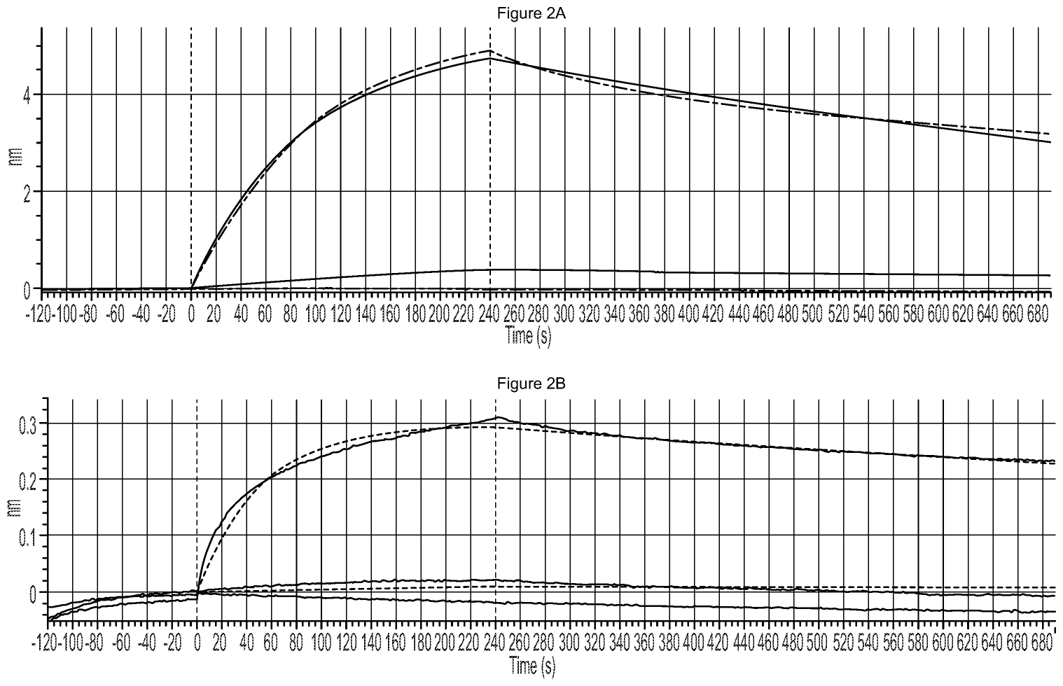

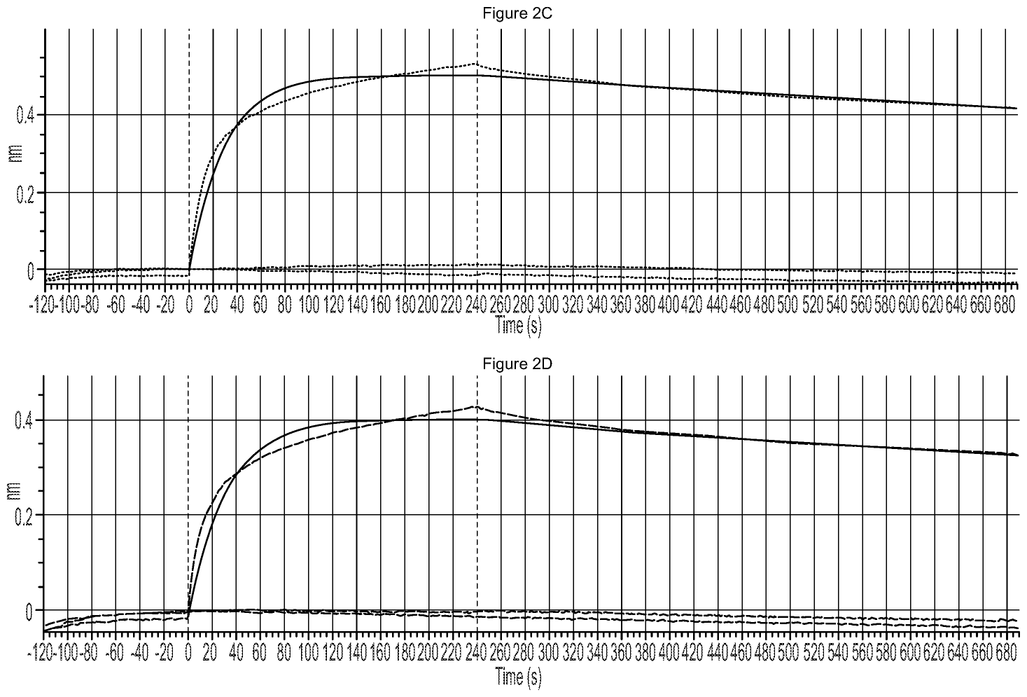

BLI is an optical analytical technique that analyzes the interference pattern of white light reflected from two surfaces: a layer of immobilized protein on a biosensor tip, and an internal reference layer. In this present case the bosensor tip was coated with streptavidin and the antibody to be tested was then bound to the streptavidin to form the layer of immobilized protein. The biosensor tip was immersed in a 100 nM, 10 nM or 0 nM solution of a RBD polypeptide in phosphate buffered saline (PBS) and the change in interference as the rBD polypeptide binds to the antibody was observed. As saturation was approached, he biosensor tip was immersed in a buffer (PBS) and the change in interference as the antibody dissociates was observed (FIG. 2A-D). From these data KD, Ka and Kd were calculated (Table 1).

TABLE 1

| | Conc | KD | ka | kdis | Full |
|---|---|---|---|---|---|
| Antibody | RBD. (nM) | (nM) | (1/Ms) | (1/s) | R² |
| Ty1-Biotin | 100 | 9.93 | 1.02E+05 | 1.01E−03 | 0.9987 |
| | 10 | 9.93 | 1.02E+05 | 1.01E−03 | 0.9987 |
| | 0 | | | | |
| MM57-biotin | 100 | 2.83 | 1.96E+05 | 5.54E−04 | 0.995 |
| | 10 | 2.83 | 1.96E+05 | 5.54E−04 | 0.995 |
| | 0 | | | | |
| R001-biotin | 100 | 1.23 | 3.44E+05 | 4.24E−04 | 0.997 |
| | 10 | 1.23 | 3.44E+05 | 4.24E−04 | 0.997 |
| | 0 | | | | |
| MM43-biotin | 100 | 1.53 | 3.05E+05 | 4.66E−04 | 0.9952 |
| | 10 | 1.53 | 3.05E+05 | 4.66E−04 | 0.9952 |
| | 0 | | | | |

### Example 4

### SARS-CoV-2 Agglutination with Nanobody Coated Beads

This experiment was carried out with Ty1 monobiotinylated nanobody coated onto three different beads: JSR Magnosphere™ MS55/Carboxyl, 550 nm beads coated with streptavidin (bead size 1); JSR Magnosphere™ MS160/Carboxyl, 1.6 μm beads coated with streptavidin (bead size 2); and Agilent 2.7 um LodeStars Streptavidin beads (bead size 3). The beads were processed as described above and suspended at 1 mg/ml in Tris buffered saline (TBS; 10 mM Tris, 150 mM NaCl, 0.05% TWEEN-20, 0.05% NaN3, pH 7.4).

The positive and negative samples were oral rinse plus nasal swab inserted. Virus had been heat inactivated by 10 minute incubation at 60° C. This partially denatures viral proteins and was expected to somewhat diminish the sensitivity of the assay as compared to a fresh patient sample. Positive and negative samples had been validated by PCR. The limit of detection in a PCR assay is about 15 virions/mL and corresponds to a cycle time (Ct) of about 38 cycles. A negative sample has a Ct >40. The positive sample used in this experiment had a Ct of 27.9.

100 μL of positive or negative sample was combined with 50, 100 or 200 μL of bead suspension of each of the three sizes in individual vials. After a 5 minute room temperature incubation, agglutination was scored by eye on a scale of 0—no agglutination, 1—minor agglutination, 2—good agglutination, 3—excellent agglutination. These results are presented in Table 2.

TABLE 2

| | Agglutination | Sample Volume | Bead Volume | |
|---|---|---|---|---|
| Sample | Score | added (μL) | added (μL) | Score |
| Positive | 1 | 100 | 100 | 3 |
| | 2 | 100 | 100 | 1 |
| | 3 | 100 | 100 | 1 |
| | 1 | 100 | 50 | 1 |
| | 2 | 100 | 50 | 1 |
| | 3 | 100 | 50 | 1 |
| | 1 | 100 | 200 | 1 |
| | 2 | 100 | 200 | 1 |
| | 3 | 100 | 200 | 2 |
| Negative | 1 | 100 | 100 | 0 |
| | 2 | 100 | 50 | 0 |
| | 3 | 100 | 200 | 0 |

The vials were randomized and presented to each of six laboratory technicians who were asked to score them for the presence or absence of a brown precipitate. All six easily picked out the 9 positive and 3 negative samples for 100% positive concordance and 100% negative concordance.

### Example 5

### SARS-CoV-2 Agglutination with Various Antibodies and Bead Size

This experiment compared each of the three bead sizes as described in Example 4 coated with each of the four antibodies characterized in Example 3. The experiment was carried out in a 96-well plate. The 96-well plate had been pretreated to block binding of virus or bead to the well surfaces by a 5 minute incubation with 0.023% PLURONIC F108 and 0.05% TWEEN-20 in TBS, after which the blocking solution was aspirated.

Then, 66 μL of the various bead suspensions (1 mg/mL in TBS) were dispensed into the wells of a black, clear- and flat-bottom 96-well plate. 33 μL of sample (oral rinse of 0.9% saline, plus nasal swab inserted, heat inactivated) was added to the beads and the plate given a gentle swirl by hand to mix. The positive sample used in this experiment had a Ct of 27.9. The beads were incubated at room temperature and allowed to settle (>5 minutes). The wells were subsequently imaged with a microarray reader focused at the bottom of the wells.

TABLE 3

| | | | Score | Score |
|---|---|---|---|---|
| | | | (Positive | (Negative |
| Antibody | Bead size | | Sample) | Sample) |
| Ty1 | 550 | nm | 3 | 0 |
| Ty1 | 1.6 | μm | 1 | 0 |
| Ty1 | 2.7 | μm | 2 | 0 |
| MM43 | 550 | nm | 0 | 0 |
| MM43 | 1.6 | μm | 0 | 0 |
| MM43 | 2.7 | μm | 1 | 0 |
| MM57 | 550 | nm | 3 | 0 |
| MM57 | 1.6 | μm | 0 | 0 |
| MM57 | 2.7 | μm | No data | No Data |
| R001 | 550 | nm | 3 | 0 |
| R001 | 1.6 | μm | 0 | 0 |
| R001 | 2.7 | μm | 2 | 0 |

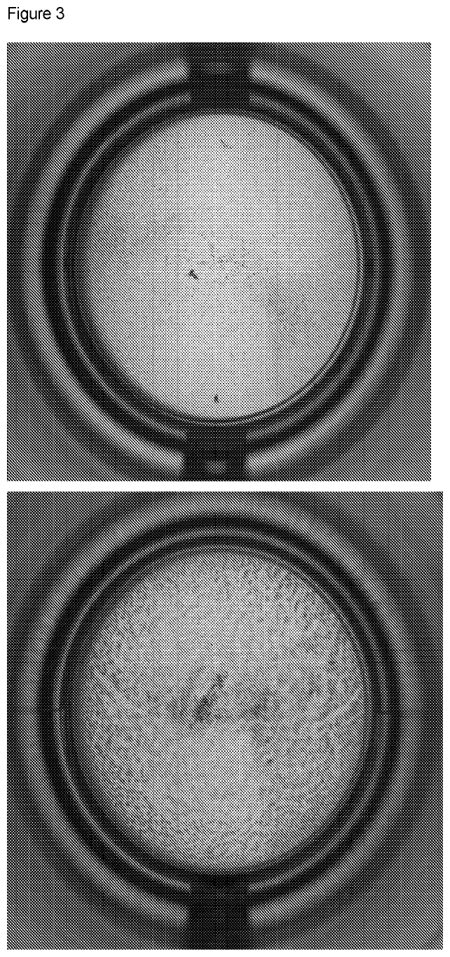

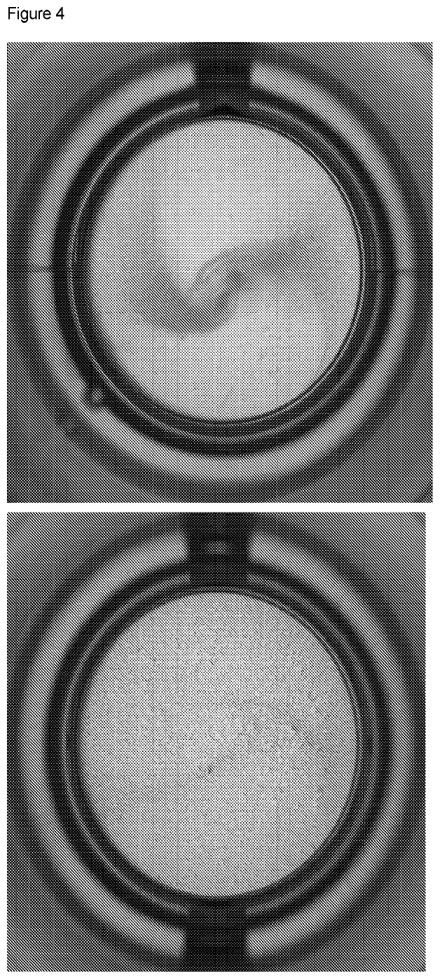

In this experiment, the Ty1 nanobody showed the least sensitivity to bead size. The MM43 antibody, which had one of the lower affinities performed consistently poorly. The images from Ty1-coated 550 nm beads, and R001-coated 2.7 μm beads, incubated with SARS-CoV-2 negative and positive samples are shown in FIGS. 3 and 4, respectively.

### Example 6

### Agglutination with Different Bead to Sample Ratios

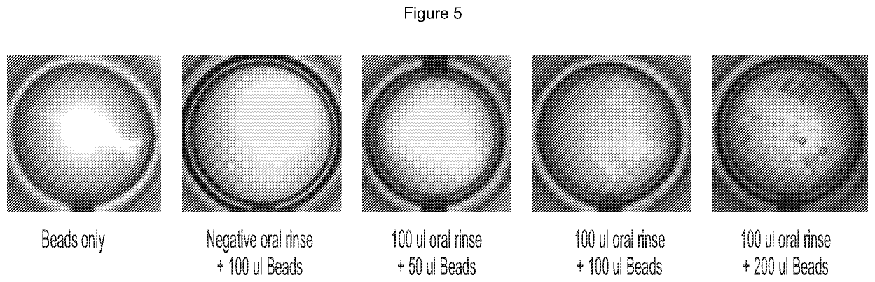

This experiment was carried out using Ty-1-coated 1.6 μm beads in similar manner to Example 5 above, using a blocked 96-well plate. The positive sample had been heat inactivated and had a PCT Ct of 27.9. Five samples were prepared: beads alone; 100 μL negative oral rinse +100 μL of bead suspension (1 mg/mL); and 100 μL positive oral rinse plus 50, 100, or 200 μL of bead suspension (that ratio of sample to beads (volume:volume) of 2:1, 1:1, and 1:2). A microarray reader was used to image the well, with the plane of focus set at the bottom of the well and the images taken after the beads had completely settled. The images are shown in FIG. 5.

### Example 7

### Agglutination with an Added Shaking Step

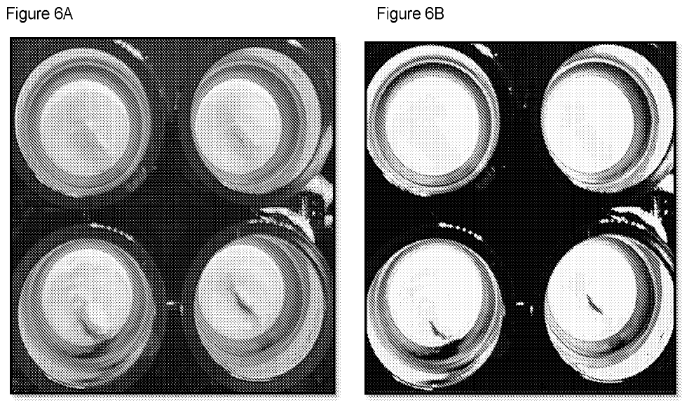

This experiment was carried out using Ty1-coated 2.7 μm beads in blocked 96-well plates. The positive (Ct of 27.9) and negative samples were oral rinse plus nasal swab inserted. One set of reactions used 66 μL of beads (1 mg/mL in TBS) and 33 μL of sample, a 2:1 ratio, and a second set used 50 μL of each, a 1:1 ratio. The reactions were incubated for about a minute before being put into a plate shaker for 5 minutes at 37° C. The shaker was set to linear shaking at about 1000 cycles/minute. The plate was removed and imaged using a smartphone camera equipped with an external 15× magnifying lens, from above, while the plate was illuminated from below. The agglutinates form a dark band near the center of the well. Image processing can be used to make the difference between positive and negative more stark. See FIGS. 6A and 6B.

### Example 8

### Titration of Bead Concentration in Agglutination Reaction

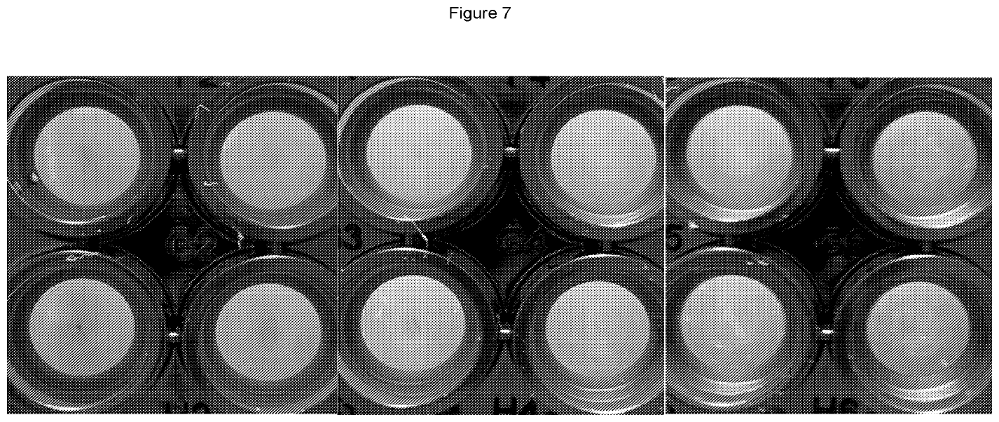

This experiment was carried out using R001-coated 2.7 μm beads in blocked 96-well plates. The positive and negative samples were oral rinse plus nasal swab inserted and the positive sample had a PCR Ct of 25.92, A six concentration bead dilution series was used having a ratio of bead content of 100:75:50:25:10:0. The well with the highest concentration of beads received 66 μL of a 2 mg/mL suspension in TBS, with each successive well in the dilution series receiving proportional less volume. The missing volume was made up with diluent of 0.9% saline (the same as the oral rinse solution). Each well received diluent, 99 μL of sample, and beads, in that order. The reaction was set up in a blocked, flat- and clear-bottomed black 96-well plate, as above. After adding the reagents the plate was allowed to sit for 5 minutes without mixing, and then placed in a plate shaker for 5 minutes at 37° C. The shaker was set to linear shaking at about 1000 cycles/minute. The plate was then removed from the plate shaker and images were obtained with a smartphone (see FIG. 7). At the highest bead concentration there is relatively little difference between the positive and negative sample, but as the bead concentration is reduced an increasingly more distinct band is formed in the centers of the positive wells, while the negative wells the beads remain in a diffuse and somewhat circular pattern.

### Example 9

### Point-of-Care Device for SARS-CoV-2 Agglutination Assay

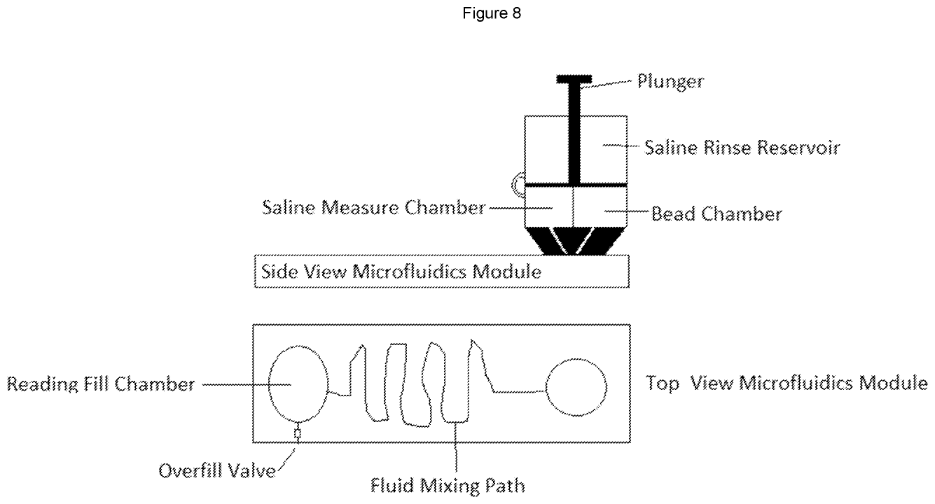

A point of care device to minimize sample handling and manipulation is pictured in FIG. 8.

An oral rinse (or similar sample) is added to the Saline Rinse Reservoir and the Plunger depressed, causing the sample to mix and be collect in the reading fill chamber. No other manipulations of the sample or assay reagents are required. Agglutination is assessed visually or with a smartphone camera and app.

The device comprise a cylinder and a Microfluidics Module. The cylinder contains a plunger and comprises three spaces and four channels. The interior volume of the cylinder above the plunger head constitutes a Saline Rinse Reservoir into which a sample is received. The interior volume of the cylinder below the plunger head is divided in two by a deformable plastic or Mylar® film. One of the spaces constitutes a bead chamber containing a suspension of anti-RBD antibody-coated beads. The other space is a Saline Measure Chamber. Its volume is less than the volume of the Saline Reservoir and the expected volume of the sample. A channel (pictured on the left side of the cylinder) allows sample to drain from the Saline Rinse Reservoir into Saline Measure Chamber and a vent channel (not pictured) allows air to escape. By filling the Saline Measure Chamber a fixed volume of sample is obtained without the user needing to measure or transfer the sample. A channel from each of the chambers leads to the Fluid Mixing Path of the Microfluidics Module. The two channels may optionally meet at an inline mixer (not pictured) before connecting to the Fluid Mixing Path. The Reading Fill Chamber is fitted with an Overfill Valve to allow air to escape as the chamber fills, but prevent liquid from exiting the chamber. The top of the Reading Fill Chamber is transparent, for example, thin clear optical polystyrene so that the presence or absence of agglutinate formation may be observed and/or photographed. The bottom of the Reading Fill Chamber may be transparent, like the top, in which case the device should be placed on a light colored background to be read (photographed). Alternatively, the bottom may be an opaque white pigmented plastic.

Once sufficient sample has drained to fill the Saline Measure Chamber, the plunger can be depress. Upon depressing the plunger, seals retaining the bead suspension and sample in their chambers are breached and the two fluids flow through the channels in the bottom of the cylinder through a T-junction, the inline mixer (if present), and Fluid Mixing Path into the Reading Fill Chamber. Mixing is also aided by the tortuosity of the Fluid Mixing Path. The device can be scaled to operate with 100 μL each of bead suspension and sample.

### Example 10

### POC—Smartphone App-Based Test

A smartphone camera is used to image the finished agglutination reaction and an app is used to read the result. The diagnostic result is based on image analysis by AI trained on positive and negative samples. This technology prevents human errors in reading the sample and makes fast, easy, accurate testing available to everyone.

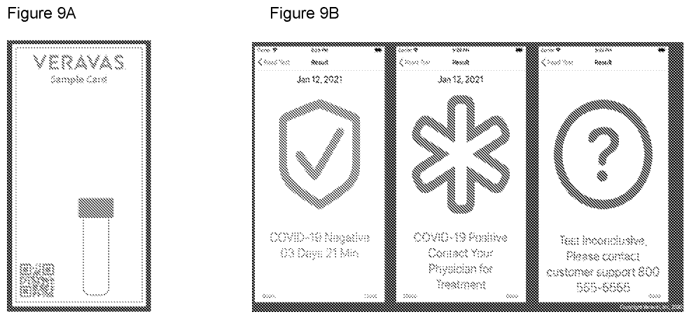

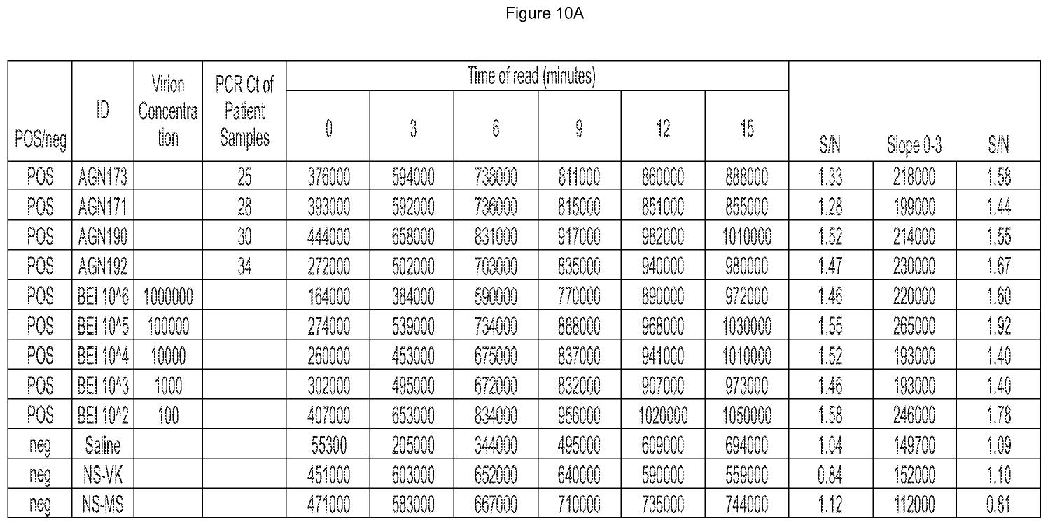

To run this POC test the following procedure is used:
1) Remove plastic spit cup from kit
2) Remove oral rinse solution bottle from kit, remove twist cap, empty entire 5 mL oral rinse into mouth, swish for 25 seconds and gargle for 5 seconds.
3) Spit the 5 mL oral rinse saliva sample into the plastic spit cup
4) Remove the capillary lancet device from the kit (saliva sample collection device) and insert it into the oral rinse saliva sample in the spit cup to draw 50 uL of saliva sample into the capillary lancet
5) Remove the agglutination reagent sealed vial from the kit and mix 10 times (inversions up/down) to mix the beads into suspension. (If a clear vial is used the user can be instructed to mix until a light brown with no dark spots or bead clumps visible).
6) Insert the capillary lancet with saliva sample into the agglutination reagent by puncturing the seal as it is inserted and insert until the capillary lancet fits snug or tight into the agglutination reagent vial to form a seal.
7) Mix the saliva sample with the agglutination reagent by inverting 10 times.
8) Remove the cap covering the dropper end on the capillary lancet device inserted into the agglutination reagent vial.
9) Place a test card with test chamber onto a flat surface, or also place test chamber onto the test card such that it covers test chamber outline or square.
10) Hold the dropper end over the test chamber and gently squeeze the bottle to add a sufficient number of drops to cover bottom of the test chamber.
11) The app will have a timer and instruct how and when to take the picture of the sample in the test chamber, in front of the test card (FIG. 9A)
12) The test result is displayed on the app (FIG. 9B)

A test result displayed on the smartphone screen may include a time code (see FIG. 9B, left panel) indicating when the person tested negative (or positive). The negative test code can serve as a badge to facilitate entry into a public gathering (for example, airline flights, work, sporting events, church, schools, etc.). Instead of, or in addition to the time code there could be a bar code or QR code. The result screens may use distinct colors for either the text and image, or the background. For example the negative result screen could use green or blue, the positive test screen could use red, or orange, or yellow; and the inconclusive test screen could use blue or black. Other color schemes using a different color for each result are possible.

### Example 11

### Confirmatory Testing of Agglutinated Virus

It is possible that coronaviruses that will cross-react with any particular anti-SARS-CoV-2 surface antigen antibody exist, and can generate a false positive result. Additionally, multiple strains of SARS-CoV-2 have been identified, and which strain an individual is infected with may be of clinical or epidemiologic interest. Thus it can be useful to confirm that the virions captured in the agglutinate are in fact SARS-CoV-2 and/or identify what strain they are. Both ends can be achieved by subjecting the captured virions to PCR (or similar) testing.

The agglutinate bound virions are heat inactivated and lysed, the beads magnetically separated, and the liquid containing the lysed virus aspirated and subjected to PCR, as usual. Alternatively, the virions are eluted by washing with Glycine pH 2.5 elution buffer. The solution is then adjusted to neutral pH, the beads magnetically separated, and the eluted purified virions aspirated and subjected to PCR, as usual.

### Example 12

### Sample Cleaning with Anti-SARS-CoV-2 Surface Antigen Antibody Coated Magnetic Beads

Saliva is not necessarily a clean substance but may contain substances from coffee, gum chewing, tobacco, food, etc. that could interfere with a PCR assay. Anti-SARS-CoV-2 surface antigen antibody coated magnetic beads offer a way to clean interference from saliva samples that interferes with PCR testing. For this application it is not essential that agglutination occurs as long as virions bind to the beads.

Anti-SARS-CoV-2 surface antigen antibody coated magnetic beads are added to saliva samples to capture the SARS-CoV-2 virions for magnetic washing with 0.9% saline to wash away these interferences. The bead bound virions are then heat inactivated and lysed, the beads magnetically separated, and the liquid containing the lysed virus aspirated and subjected to PCR, as usual.

Alternatively, the virions are eluted by washing with Glycine pH 2.5 elution buffer. The solution is then adjusted to neutral pH, the beads magnetically separated, and the eluted purified virions aspirated and subjected to PCR, as usual.

### Example 13

### Model Protocols for a SARS-CoV-2 Aggregation/Agglutination Assay

Method Overview: Paramagnetic microparticles (PMPs) coated with antibodies against SARS-CoV-2 spike-RDB proteins are introduced to a human sample from a nasal swab mixed into saline. The PMPs react by clumping together. The clumped PMPs are then imaged and counted, for example, by a BioTek Cytation 5 microscopic cell counter. Clumps are counted in a size gate. Counted clumps or the rate of increase of clumps over time is proportional to the presence of SARS-CoV-2 virions in the sample.

### Materials:

1) Streptavidin Coated PMPs (1.6 μm diameter) conjugated with biotinylated MM57 anti-RBD monoclonal antibody (see Example 1).
2) Nasal Collection Swabs (Copan PN502CS01 Copan Diagnostics Inc, 26055 Jefferson Ave, Murrieta, CA 92562).
3) Saline (0.09% NaCl in purified water).
4) Cytation 5 Cell Imaging Multi-Mode Reader.
5) Alpaqua Catalyst™ 96, 96 well Slotted Ring Magnet Plate SKU: A000550 (Alpaqua Inc. 100 Cummings Center, Suite #424A, Beverly, MA 01915).
6) Reaction plate (Corning PN353910 Corning Inc, 1 River Front Plaza Corning NY 14831)
7) Half Area Reading Plate (Greiner PN675090 Greiner BioOne GMBH Maybach St 2, 72636 Frickenhausen, Germany).
8) TTA: Tris Buffered Saline with 0.05% Tween 20 and 0.05% sodium azide.
   The protocol can be adapted to use equivalent reagents from other suppliers.

### Protocol 3 Collection of Samples

1) Give sterile sample swab to patient
2) Instruct patient to gently swab anterior nasal canal by rotating swab 5 times against nasal canal.
3) Place swab directly into 3 mL saline in 15 mL collection tube
4) Cap tube and vortex
5) Remove liquid to secondary collection tube and centrifuge
6) Remove centrifuged clear liquid for testing

### Test Protocol

1) Add 50 μL centrifuged (10 min at 21380 rcf) patient sample to reaction plate
2) Add 50 μL antibody-conjugated PMPs at 1 mg/mL in TTA to each 50 μL patient sample in reaction plate
3) Place at 30° C. in oscillating shaker (500 RPM) for 30 minutes
4) Place reaction plate on 96 place magnet for 5 minutes
5) Condition Reading plate by adding 15 μL TTA To each well
6) Re-suspend reaction plate pellet using pipette mixing
7) Add 10 μL of the resuspended PMPs from step 6 to half area reading plate
8) Put reading plate into Cytation 5 and read kinetically for 15 minutes at 3 minute intervals
9) Export the following OSA size gates data
   a. 2-30 microns (preferred)
   b. 15-20 microns
   c. 25-30 microns
   d. OR Calculate Slope from data measurements at 0 and 3 minutes.
10) Use Saline signal+15% as Positive cutoff
11) Positive samples have signal>Saline+15%

### Example 14

### SARS-CoV-2 Assay Result

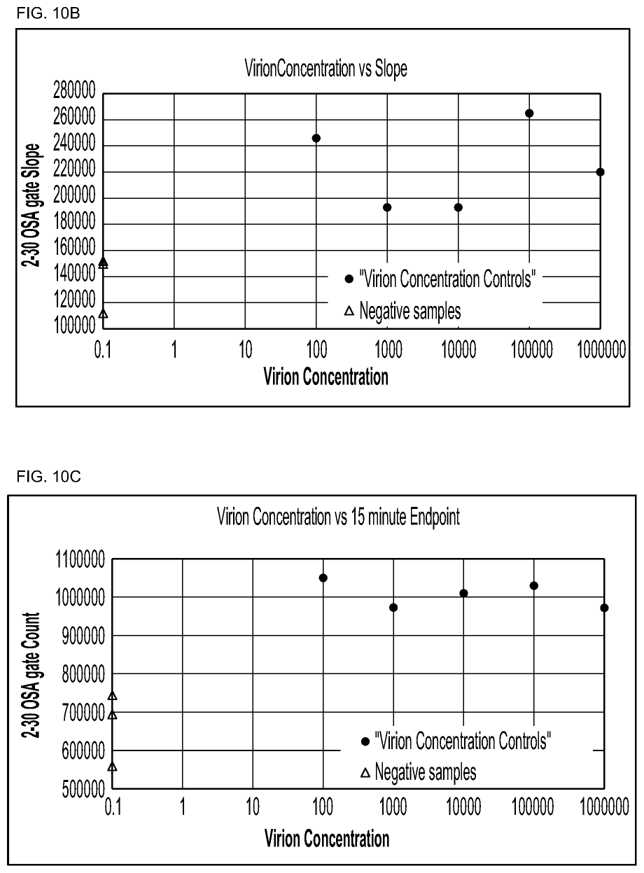

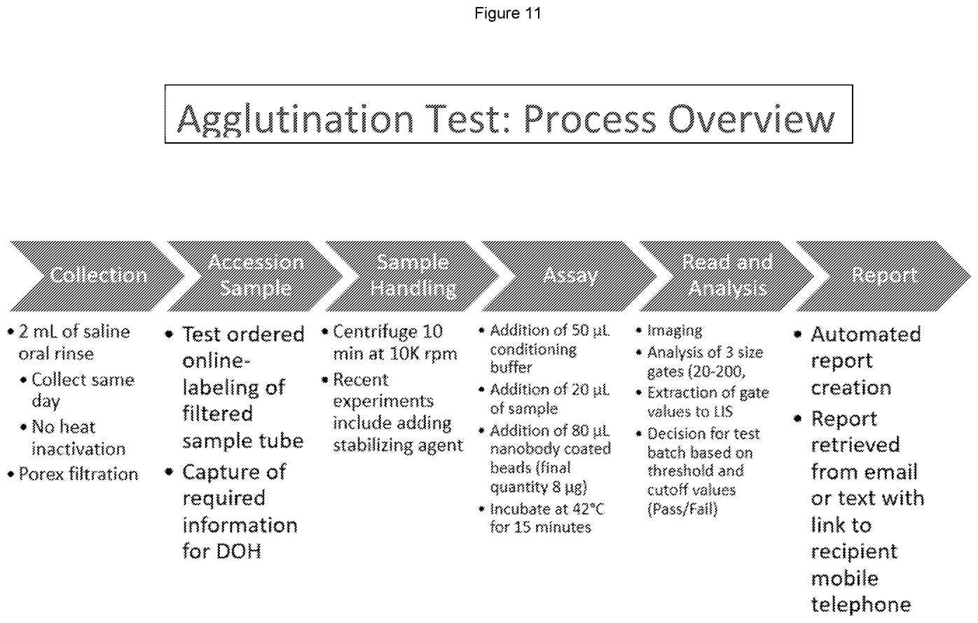

Positive and negative patient saliva samples and serially diluted SARS-CoV-2 virus were assayed essentially as described in Example 13 (above). The data collection gate was set to 2-30 microns and read at 3 minute increments from 0 to 15 minutes (FIG. 10A). The saliva samples from four SARS-CoV-2-infected patients with PCT assay results were obtained from Access Genetics (Eden Prairie, MN). SARS-Related Coronavirus 2, Isolate USA-WA1/2020, Gamma-Irradiated (BEI Resources, Manassas, VA) was used to prepare 10-fold serial dilutions of virions in saline for 106 to 102 virions/mL.

Object sum area (OSA) readings (a 2-dimensional projection of the beads) for the three negative samples (one saline sample and two saliva samples from uninfected volunteers) were averaged and the cutoff between positive and negative set at 115% of the average OSA reading for the negative samples for the 15 minute endpoint (665667) and the slope over 0 to 3 minutes (137900). The signal to noise ratio (S/N; FIG. 10A) between the OSA reading for each positive sample and the negative sample average was also determined, showing positive and negative could be easily discriminated. The OSA readings for each of the samples were plotted for both the 15 minute endpoint (FIG. 10B) and the slope over 0 to 3 minutes (FIG. 10C). (The negative samples were arbitrarily plotted at 0.1 virion per mL as there is no true zero on a log scale). Again, one can readily discriminate between positive and negative samples. The results of the assay are qualitative and not quantitative. There was not a correlation between the virion concentration (or PCR cycle number) and the magnitude of the reading.

### Example 15

### SARS-CoV-2 Assay Results (Nasal Swab)

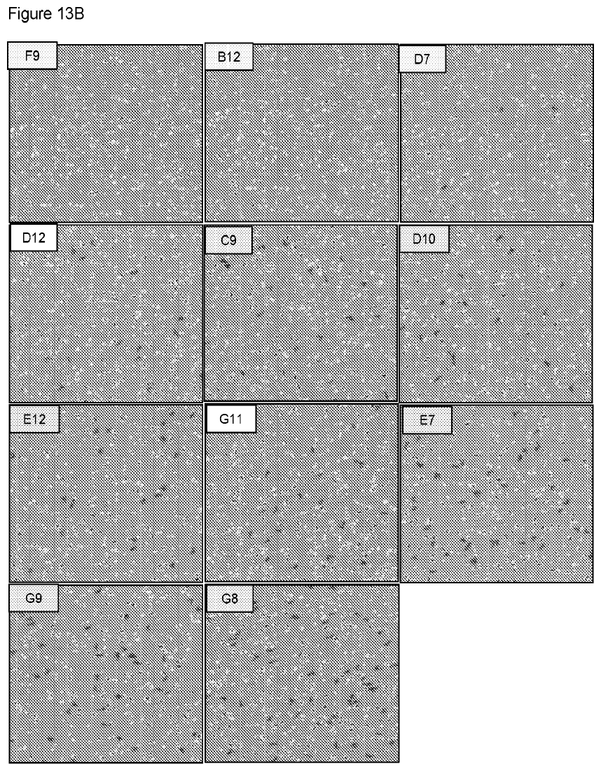

The assay was run under conditions that varied from the above example. Nasal swab samples from SARS-CoV-2 infected and uninfected persons (as judged by PCR), RBD-coated latex beads, and titrated virion samples, were assayed. 20 μL of nasal swab sample was added to 50 μL in a 96-well plate. Then, 80 μL of PMP conjugated with anti-RBD monoclonal antibody was added to each well and incubated for 5 minutes at 37 ° C. on an external plate heater. The place was inserted into a Biotek Cytation 5 plate reader at ambient temperature and OSA readings collected in a 9-99 micron particle diameter window (FIGS. 13A-B). Slope was calculated over the interval of 17-21 minutes.

As seen in Table 4 (below), a slope of less than about 1000 correlated with a negative sample. Although 10 virions per sample was below detection by PCR, it gave a positive signal in this agglutination assay.

TABLE 4
OSA and Calculated Slope of the various samples and controls

| | | PCR | OSA 9-99 μm | Slope |
|---|---|---|---|---|
| Sample | | cycle No. | (at 21 | (17-21 |
| Well | Description | (Ct) | minutes) | min) |
| F9 | Nasal swab in PBS | Negative* | 2900 | 348 |
| B12 | Nasal swab in saline | Negative | 4760 | 373 |
| D7 | Nasal swab in saline** | Negative | 11400 | 940 |
| E12 | 10 virions in saline/well | Negative | 23500 | 2375 |
| C9 | 10³ virions in saline/well | 38.9 | 1740 | 1725 |
| E7 | 10⁶ virions in saline/well | 28.7 | 29600 | 2400 |
| G11 | 50 nm latex beads coated | N/A | 27400 | 2350 |
| | with RBD:BSA 1:1 | | | |
| | (10 pg/well) | | | |
| D12 | 50 nm latex beads coated | N/A | 12300 | 933 |
| | with RBD (10 pg/well) | | | |
| D10 | Nasal swab in saline** | 37.33 | 20500 | 1850 |
| G9 | Nasal swab in saline** | 15.84 | 36900 | 2550 |
| G8 | Nasal swab in saline** | 29.69 | 38600 | 3300 |

*A negative result by PCR is taken as a Ct ≥ 40
**PCR confirmed patient sample from Access Genentics

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of detecting SARS-CoV-2 infection in one or more individuals comprising:
a) individually combining anti-SARS-CoV-2 surface antigen antibody-coated beads with a saliva sample from each of said one or more individuals to form an admixture corresponding to each individual;
b) mixing the beads and saliva sample of each individual's admixture;
c) incubating the admixture(s); and
d) detecting whether agglutination has occurred in each individual's admixture.

2. The method of claim 1, wherein the surface antigen is an S1 or S2 spike protein.

3. The method of claim 2, wherein the antibody recognizes a receptor binding domain or N- terminal domain (NTD) of the S1 spike protein.

4. The method of claim 2, wherein the saliva sample is comprised in an oral rinse fluid.

5. The method of claim 4, wherein the oral rinse fluid is obtained by having each of the one or more individuals swish and gargle a saline solution in their oral cavity and then expectorate into a collection vessel, whereby the oral rinse fluid is obtained,

6. The method of claim 5, wherein the anti-SARS-CoV-2 surface antigen antibody-coated beads are colored and have a dark or intense shade.

7. The method of claim 6, wherein the anti-SARS-CoV-2 surface antigen antibody-coated beads are magnetic.

8. The method of claim 7, wherein the anti-SARS-CoV-2 surface antigen antibody-coated beads are streptavidinated.

9. The method of claim 8, wherein the antibody is biotinylated and a biotin moiety is bound to streptavidin.

10. The method of claim 9, wherein the anti-SARS-CoV-2 surface antigen antibody-coated beads have a diameter of from 1.3 to 1.9 pm.

11. The method of claim 10, wherein the anti-SARS-CoV-2 surface antigen antibody-coated beads have a diameter of from 1.6 pm.

12. The method of claim 9, wherein the anti-SARS-CoV-2 surface antigen antibody-coated beads have a diameter of 2.7 pm.

13. The method of claim 12, wherein the anti-SARS-CoV-2 surface antigen antibody-coated beads comprise from 25 to 35 pg of antibody/mg of beads.

14. The method of claim 13, wherein the anti-SARS-CoV-2 surface antigen antibody-coated beads comprise 30 pg of antibody/mg of beads.

15. The method of claim 14, wherein the incubating is at room temperature.

16. The method of claim 14, wherein the incubating is at 37° C.

17. The method of claim 16, wherein each admixture is incubated for 2-5 minutes.

18. The method of claim 17, wherein the detecting comprises visual observation.

19. The method of claim 17, wherein the detecting comprises use of a camera.

20. The method of claim 17, wherein the detecting comprises use of an optical density meter.

21. The method of claim 17, comprising transferring an aliquot from each individual's admixture to a well of a multiwell plate after the mixing or incubating step and detecting whether agglutination has occurred for each individual's admixture.

22. The method of claim 21 wherein the detecting comprises use of a microplate reader.

23. The method of claim 21 , wherein the detecting comprises use of a microarray digital reader.

24. The method of claim 23, further comprising adding a viscosifying agent to the admixture after the incubating step but before the transferring step.

25. The method of claim 24, wherein the viscosifying agent is FICOLL.

26. The method of claim 25, further comprising:
a) combining samples containing known amounts of virions with anti-SARS-CoV-2 surface antigen antibody-coated beads to form a standard curve admixture corresponding to each amount of virions,
b) mixing the beads and virion samples of each standard curve admixture,
c) incubating the standard curve admixtures, and
d) transferring each standard curve admixture to a well of the multiwell plate after the mixing or incubating step and detecting whether agglutination has occurred for each standard curve admixture;
wherein the standard curve admixtures serve as a calibration curve from which the amount of virus in the saliva samples can be quantitated.

27. A method of detecting SARS-CoV-2 infection in one or more individuals comprising: detecting whether agglutination has occurred in an incubated admixture corresponding to each individual, wherein each admixture includes anti-SARS-CoV-2 surface antigen antibody-coated beads and a saliva sample from one individual.

28. The method of claim 27, wherein the incubated admixture was incubated for 2-5 minutes room temperature.

29. The method of claim 27, wherein the incubated admixture is simultaneously shaken for 1-5 minutes.

30. The method of claim 29, wherein the incubation is at 37° C.

31. The method of claim 30, wherein the anti-SARS-CoV-2 surface antigen antibody is an alpaca-derived nanobody.

32. The method of claim 31, wherein the nanobody is Ty1.
